(19) United States
(12) Patent Application Publication
(10) Pub. No.: US 2004/0243129 A1
(43) Pub. Date: Dec. 2, 2004

(54) Double helical threaded bone screw

(76) Inventors: Missoum Moumene, Newton, MA (US); Therese Kelley, Scituate, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/446,626

(22) Filed: May 28, 2003

(51) Int. Cl. A61B 17/86

(52) U.S. Cl. 606/73; 606/72

(57) ABSTRACT

A bone screw is provided having a head adapted to mate with a driver tool, and a shank formed from first and second helical threads that extend distally from the head around a core. The core defines a minor diameter which, along at least a portion of the shank, is preferably equal to or less than a thickness of the threads between the proximal and distal facing flanks. The bone screw is particularly advantageous in that the shape of the threads provides a high pullout strength, and results in a relatively small core diameter, thereby reducing or eliminating the risk of damage to the bone. The small core diameter also provides a sufficiently flexible screw with an adequate bending strength to handle forces acting on the screw, and to prevent breakage of the screw or the screw head.

### FIELD OF THE INVENTION

The present invention relates to bone screws, and in particular, to a bone screw having improved physical and mechanical properties.

### BACKGROUND OF THE INVENTION

Bone screws are used for a variety of medical purposes, including to correct spinal pathologies, deformities, and trauma. Spinal bone screws are loaded with axial, distractive, and compressive forces, and with subsequent cyclically loaded forces applied through the patient's natural movement. Thus, spinal bone screws must be sufficiently strong, while at the same time they must be designed to minimize the potential damage to the bone.

Conventional bone screws are typically made from a cylindrical or tapered core having a helical thread with either a variable or a constant major diameter extending along the entire length of the screw. The helical shape of the threads cuts a path into the bone as the screw rotates, and prevents the screw from being axially pulled out of the bone. Thus, threads having relatively deep flanks and/or a small core diameter will increase the pull-out strength of the screw. Conventional bone screws, however, typically require a relatively large core diameter to resist all forces on the screw to permit the screw to have an uncompromised retention in the bone. Moreover, a relatively large core diameter is often necessary to withstand high torque without shearing or otherwise failing. A thick core can, however, displace enough bone to cause the bone to split or otherwise become damaged. Screws with thicker cores also tend to result in a substantially rigid screw, which can be undesirable as the screw needs an adequate bending strength to react to the biomechanical forces acting on the screw without damaging adjacent bone or breaking.

Accordingly, there is a need for an improved bone screw having a high pull-out strength, yet that has an adequate bending strength and that causes minimum damage to the bone.

### SUMMARY OF THE INVENTION

The present invention generally provides a bone screw having a head, a shank, a major diameter, and a minor diameter. First and second helical threads having a root and a crest extend around the length of the shank and define a thread thickness extending between proximal and distal facing flanks. The thread thickness adjacent the root of the threads can be substantially constant along the entire length of the shank, but it is preferably equal to or greater than the minor diameter of the shank along at least a portion of the length of the shank. In an exemplary embodiment, the thread thickness adjacent the root of the threads is equal to or greater than a minor diameter of the shank at a distal end of the shank. This is particularly advantageous because the small core diameter provides a sufficiently flexible screw with an adequate bending strength to handle biomechanical forces, and the threads provide a high pullout strength.

In one embodiment, the thread thickness can vary between the root and the crest of each thread. By way of non-limiting example, the proximal and distal facing flanks of each thread can converge toward one another at an angle from the root to the crest of the threads. Alternatively, the proximal and distal facing flanks can be parallel to one another along a first, major portion of the flanks, while converging toward one another at an outer-most crest of each thread to form a beveled edge. In an exemplary embodiment, the threads include a crest having a width which forms a flat surface extending between the proximal and distal facing flanks. The width preferably remains substantially constant along the length of the shank.

In another embodiment, the shank of the bone screw has a minor diameter that can be substantially constant along a length of the shank, or that can vary along the length of the shank. In an exemplary embodiment, at least a portion of the minor diameter of the shank decreases in a proximal-to-distal direction to form a tapered portion. While the minor diameter varies, the major diameter of the screw is preferably substantially constant along a substantial length of the screw.

In other aspects, the threads of the bone screw define a bone-receiving area between adjacent flanks that is preferably adapted to seat a relatively large amount of bone, at least compared to conventional bone screws. The bone-receiving area can have a volume that is at least about 20%, and more preferably is about 30%, of a volume of the shank defined by the major diameter and the length of the screw. This is particularly advantageous in that it provides a bone screw having a high pull out strength.

### BRIEF DESCRIPTION OF THE DRAWINGS

The invention will be more fully understood from the following detailed description taken in conjunction with the accompanying drawings, in which:

FIG. 1A is a perspective view of a bone screw according to one embodiment of the present invention having a constant core diameter and having substantially parallel flanks;

FIG. 1B is a cross-sectional view of the bone screw shown in FIG. 1A;

FIG. 1C is a cross-sectional view of the bone screw shown in FIG. 1A having a box disposed there around representing the total volume;

FIG. 1D is a cross-sectional view of the bone screw shown in FIG. 1A having shading representing the volume of bone to be occupied within the bone-receiving area of the bone screw;

FIG. 2A is a perspective view of another embodiment of a bone screw according to the present invention having a one-quarter tapered shank and having substantially converging flanks;

FIG. 2B is a cross-sectional view of the bone screw shown in FIG. 2A;

FIG. 3A is a perspective view of another embodiment of a bone screw having a one-half tapered shank;

FIG. 3B is a cross-sectional view of the bone screw shown in FIG. 3A;

FIG. 4A is a perspective view of another embodiment of a bone screw having a fully tapered shank;

FIG. 4B is a cross-sectional view of the bone screw shown in FIG. 4A; and

FIG. 5 is a chart illustrating the volume of bone occupied by several different bone screws according to the present invention compared to the volume of bone occupied by a conventional prior art screw.

### DETAILED DESCRIPTION OF THE INVENTION

In general, the present invention provides a bone screw having a head adapted to mate with a driver tool, and a shank that includes first and second helical threads. Each thread includes a proximal facing flank and a distal facing flank, and each threads is preferably offset approximately 180° with respect to the other. Each thread begins at the head of the screw, or at a position just distal to the head, and terminates at an apex that forms distal tip of the screw, or at a position just proximal to the apex of the screw. The two helical threads form a core having a diameter which, along at least a portion of the shank, is preferably equal to or less than a thickness of the threads between the proximal and distal facing flanks. The bone screw is particularly advantageous in that the shape of the threads provides a high pullout strength, and results in a bone screw having a relatively small minor diameter, thereby reducing or eliminating the risk of damage to the bone. The small minor diameter also provides a sufficiently flexible screw with an adequate bending strength to handle forces acting on the screw, and to prevent breakage of the screw or the screw head. The double helical threads also allow the screw to advance more quickly into bone.

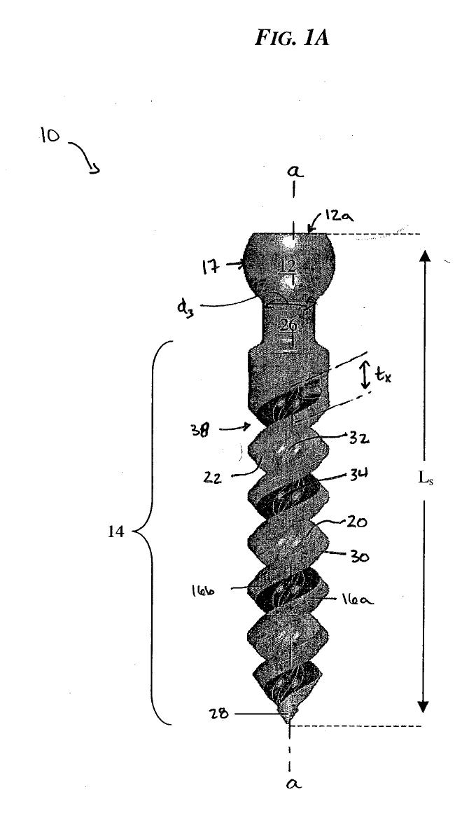

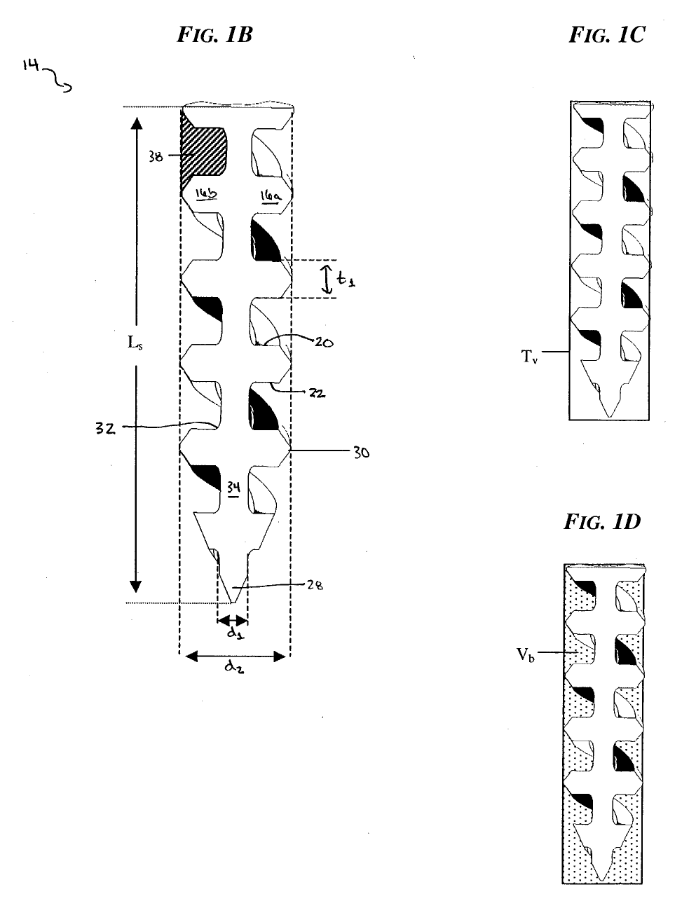

FIGS. 1A and 1B illustrate one embodiment of a bone screw 10 according to the present invention. As shown, the bone screw 10 includes a proximal head 12 having a shank 14 that includes first and second helical threads 16a, 16b extending distally therefrom and offset approximately 180° with respect to each other.

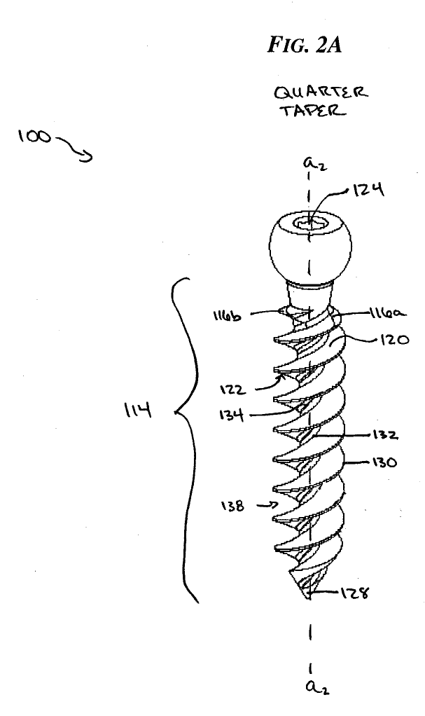

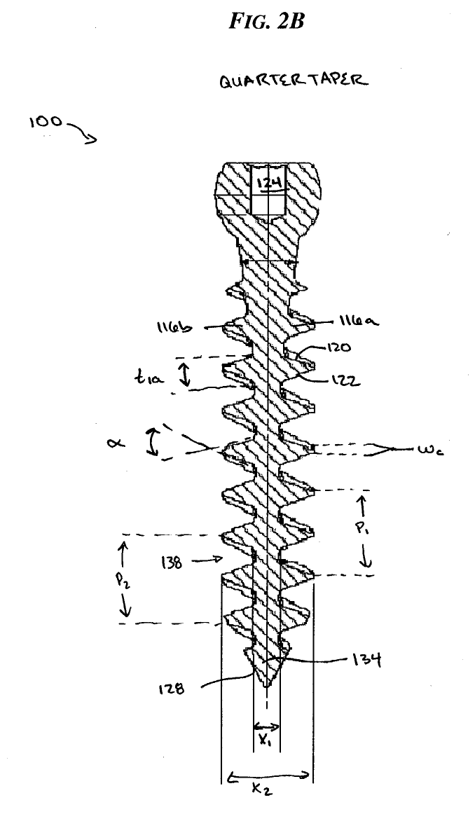

The head 12 of the bone screw 10 can have a variety of configurations, and can be adapted for a variety of uses. As shown in FIG. 1A, the head 12 of the bone screw 10 has a substantially spherical mating surface 17, but includes a flattened proximal surface 12a. A driver-receiving element 124 (shown in FIG. 2A) is formed in the proximal surface 12a of the head 12 and is adapted to mate to a driver tool for driving the bone screw 10 into bone. The driver-receiving element 124 can have a variety of configurations. As shown in FIG. 2A, the driver-receiving element 124 is in the form of a hexagonal socket for receiving a hexagonally-shaped driver member. A person skilled in the art will appreciate that a variety of driver-receiving elements can be used, and that the head 12 of the bone screw 10 can have virtually any configuration.

The threads 16a, 16b that form the shank 14 of the bone screw 10 can extend distally from the head 12, or, depending on the type of bone screw intended, the threads 16a, 16b can start at a position spaced apart from the head 12 such that the bone screw 10 includes a thread-free shank portion 26. As shown in FIGS. 1A and 1B, the bone screw 10 is a polyaxial screw, and thus the thread-free shank portion 26 allows the screw 10 to rotate within a screw-receiving bore formed in another medical implant, such as a rod-receiving head of a spinal implant. The thread-free shank portion 26 can also be effective to provide some rigidity to the head 12 of the bone screw 10 to minimize any risk of the head 12 breaking apart from the shank 14 during use of the screw 10. The thread-free portion 26 of the shank 14 can have any diameter d₃, but preferably the diameter d₃ of the thread-free portion 26 is the same as or less than a major diameter d₂ of the shank 14, which will be discussed in more detail below.

As noted above, the helical threads 16a, 16b preferably start at a position approximately 180° apart from one another on the shaft and terminate at or adjacent to an apex 28 that forms the distal tip of the screw 10. The apex 28 can have a variety of configurations. By way of non-limiting example, the apex 28 can be in the form of a cone-type or gimlet-type tip. As shown in FIG. 1A, the apex 28 of the screw 10 is in the form of a gimlet tip, wherein the threads 16a, 16b extend to and merge at the distal tip of the screw 10. With cone-type tips, the threads 16a, 16b terminate at a position just proximal to the distal tip core of the screw is formed into a solid, cone-like structure. A person skilled in the art will appreciate that either tip can be used, or alternatively the apex 28 can have a variety of other configurations.

Still referring to FIGS. 1A and 1B, the threads 16a, 16b also include a thickness tthat is defined by the distance between a proximal facing flank 20 and a distal facing flank 22. The thickness t₁ can vary along the length Lₛ of the shank 14, as well as between the root 32 and a crest 30 of each thread 16. As shown in FIG. 1B, the thickness t₁ of the threads 16a, 16b is substantially constant along the length Lₛ of the shank 14, as well as between the root 32 and the crest 30 of the threads 16a, 16b. This can be achieved by forming proximal and distal facing flanks 20, 22 that are substantially parallel to one another between a majority of the flank 20, 22 extending between the root 32 and the crest 30 of the threads 16a, 16b. While a major portion of the proximal and distal facing flanks 20, 22 are parallel to one another, the threads 16a, 16b can include a beveled crest 30 formed from an outer-most portion of the proximal and distal facing flanks 20, 22 that converge toward one another.

In an alternative embodiment, shown in FIGS. 2A-4B, the proximal and distal facing flanks 120, 122, 220, 222, 320, 322 can converge at an angle toward one another, preferably at substantially the same angle, to meet at the crest 130, 230, 330. While the flanks 120, 122, 220, 222, 320, 322 are disposed at a converging angle toward one another, the thickness t₁ₐ, t_{1b}, t_{1c} of the threads 116, 216, 316 can still remain constant along a substantial length of the shaft 114, 214, 314. The thickness t₁ₐ, t_{1b}, t_{1c} only varies between the root and the crest 130, 230, 330 of the threads, decreasing gradually from root to crest 130, 230, 330.

The crest 30, 130, 230, 330 of the threads 16a, 16b, 116, 216, 316 can have a variety of shapes and sizes, but preferably the crest 30, 130, 230, 330 forms either a sharp edge on the threads, as shown in FIGS. 1A and 1B, or the crest 130, 230, 330 has a flat edge with a small width w_{c} (shown in FIG. 2B) that remains substantially constant along the length of the shank 14. The width w_{c} is the distance between the proximal and distal facing flanks 120, 122. In an exemplary embodiment, the width w_{c} of the crest 130, 230, 330 is in the range of about 0.15 to 0.30 mm, and more preferably is about 0.2 mm.

Referring back to FIG. 1B, the core 34 forms the base for the root 32 of the threads 16a, 16b and defines a minor diameter d₁ of the bone screw 10. The bone screw 10 also includes a major diameter d₂ which is defined by the distance between crests 30 of the threads 16a, 16b. The distance between the root 32 and the crest 30 of the threads 16a, 16b is the same as the difference between the minor diameter d₁ and major diameter d₂. The minor diameter d₁ of the screw 10 can be substantially constant, or it can vary along the length Lₛ of the shank 14. The minor diameter d₁, along at least a portion of the shank 14 should, however, be equal to or less than the difference between the major and minor diameters d₁, d₂ of the bone screw 10. In an exemplary embodiment, the minor diameter d₁ is less than the thickness t₁ of the threads 16a, 16b along at least a portion of the length Lₛ of the shank 14. More preferably, at least the distal portion of the shank 34 has a minor diameter d₁ that is equal to or less than the thickness t₁ of the threads 16a, 16b and/or the difference between the major and minor diameters d₁, d₂ of the bone screw 10. As shown in FIG. 1B, the core 34 has a minor diameter d₁ that is substantially constant along the length Lₛ of the shank 14, with the exception of a distal portion of the core 34 that can taper to the terminate the threads, as well as to form the apex 28 of the screw 10. Moreover, the minor diameter d₁ is substantially the same as the thickness t₁ of the threads 16a, 16b. While the minor diameter d₁ of the core 34 can vary, the major diameter d₂ of the shank 14, in an exemplary embodiment, is constant along the length Lₛ of the shank 14, again with the exception of a distal portion of the core 34 that can taper to terminate the threads, as well as to form the apex 28 of the screw 10.

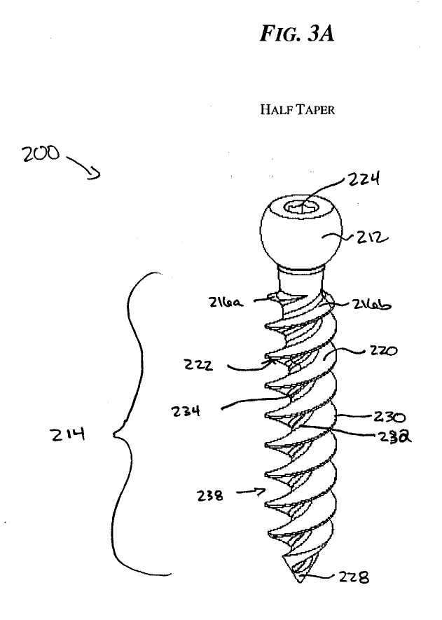

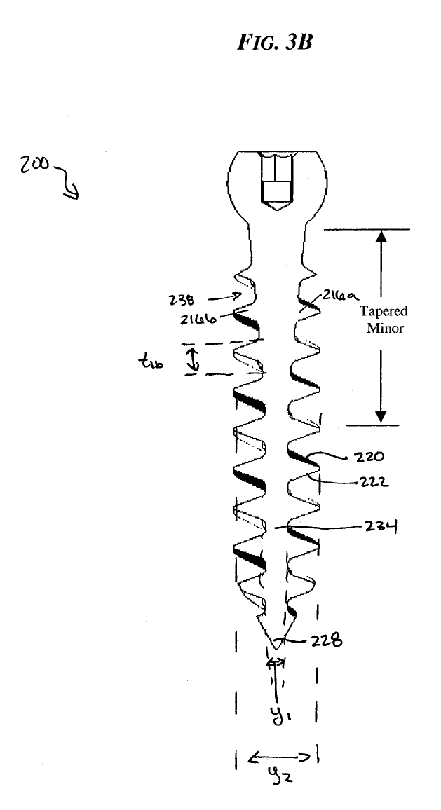

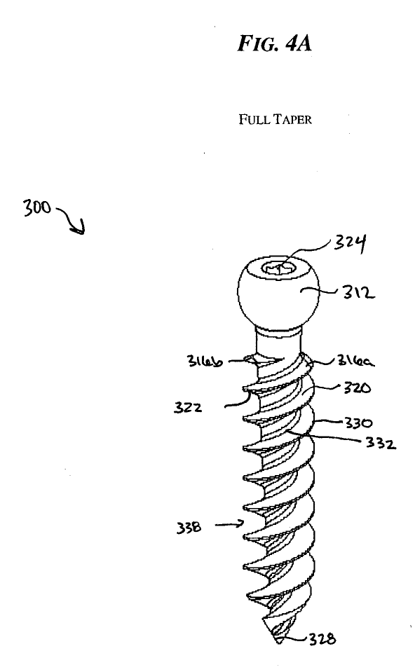

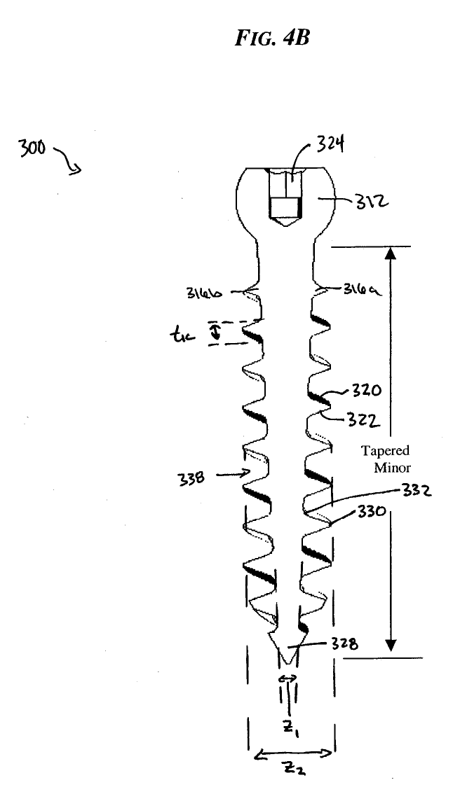

FIGS. 2A-4B illustrate alternative embodiments of a screw 100, 200, 300 having a core 134, 234, 334 with a minor diameter x₁, y₁, z₁, that increases in a distal-to-proximal direction along at least a portion of the shank 114, 214, 314 to form a tapered portion. For convenience, the prefix 1, 2 or 3 is added to the reference numbers used in FIGS. 1A and 1B to refer to corresponding parts shown in FIGS. 2A-4B. FIGS. 2A and 2B illustrate a bone screw 100 having a core 134 with a minor diameter x₁ that is substantially constant along the distal three-quarters of the shank 134, and that is tapered in a proximal-to-distal direction at the top one-quarter of the shank 134 to form a quarter tapered screw 100. While a portion of the shank 134 is tapered, the major diameter x₂ is preferably substantially constant along the length of the shank 114, with the exception of a distal portion that converges toward the apex 128. FIGS. 3A and 3B also illustrate a bone screw 200 having a tapered shank 214. The minor diameter y₁, however, is tapered along the proximal half of the shank 14, while the minor diameter y₁ remains constant along the distal half of the shank 14 to form a half tapered screw 200. FIGS. 4A and 4B illustrate a screw 300 having a minor diameter z₁ that is tapered along the full length of the shank 314. In each of the embodiments shown in FIGS. 1A-4B, the major diameter d₂, x₂, y₂, Z₂ of the screw 10, 100, 200, 300 remains substantially constant along a substantial portion of the shaft 14, 114, 214, 314. This is effective to provide a proximal facing flank having a relatively large surface area to prevent pull-out of the screw 10, 100, 200, 300.

The threads 16a, 16b, 116a, 116b, 216a, 216b, 316a, 316b of the bone screws 10, 100, 200, 300 can also have a pitch P that varies depending upon the requirements of a given screw. Referring to FIG. 2B, the pitch is determined by the distance between the threads 116a, 116b on one helix, thus the bone screw 100 can have a first pitch P₁ for the first thread 116a and a second pitch P₂ for the second thread 116b. In an exemplary embodiment, the pitch P₁, P₂ for each thread 116a, 116b is in the range of about 4 mm to 8 mm, and more preferably is about 6 mm with respect to the longitudinal axis a₂.

The bone screws 10, 100, 200, 300 of the present invention further include a bone-receiving area 38, 138, 238, 338 that is defined by a distance tₓ (FIG. 1A) between the threads 16a, 16b, 116, 216, 316 and the area adjacent to the core 34, 134, 234, 334. With reference to FIG. 1A, while the distance tₓ between the threads 16a, 16b can vary, the distance tₓ is preferably constant along the entire length Lₛ of the screw 10. As a result, the bone-receiving area 38 between the threads 16a, 16b also remains substantially constant. In FIG. 1B, the bone-receiving area 38 is shaded to show the area that is occupied by bone when the screw 10 is implanted. It is desirable to have a screw with a relatively large bone-receiving area 38 to increase the pull-out strength of the screw. A large bone-receiving area will also minimize the risk of damage to the bone since less bone will be displaced during insertion of the screw.

When the screw 10 is disposed within bone, the bone-receiving area receives or seats a particular volume of bone V_{b}, which is represented by the shaded area shown in FIG. 1D. In an exemplary embodiment, the volume of bone V_{b} received by the bone-receiving area 38 is at least about 50%, and more preferably about 60%, of a total volume Tᵥ. The total volume Tᵥ is shown in FIG. 1C and can be determined based on the major diameter d₂ and the length Lₛ of the shank 14. While the bone-receiving area will receive or seat a particular volume of bone V_{b}, the threads 16a, 16b and core 34 of the shank 14, conversely, will displace or occupy a certain volume of bone. The volume or amount of bone displaced by the shank 14 is equivalent to the volume of the shank itself, which hereinafter referred to as the shank volume. The shank volume is the difference between the total volume Tᵥ and the volume of bone V_{b} receives by the bone-receiving area 38. Since the volume of bone V_{b} received by the bone-receiving area 38 is preferably at least about 50%, and more preferably is about 60% of the total volume Tᵥ, the shank volume (e.g., the volume of bone displaced by the shank) is about 50% or less of the total volume Tᵥ, and more preferably is about 40% or less of the total volume Tᵥ when the shank 14 is disposed within bone.

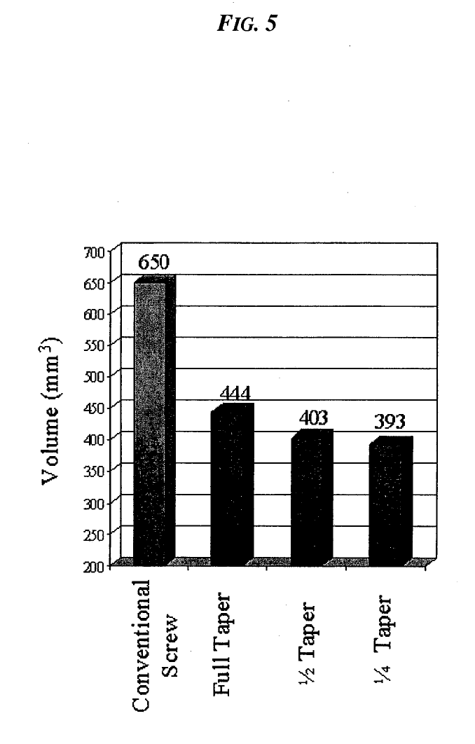

FIG. 5 illustrates the differences between three bone screws in accordance with the present invention when compared to a conventional bone screw. As indicated above, the shank volume for any given screw can be calculated based on the actual size of the screw shank itself, taking into considering certain factors which include the major and minor diameters, and the pitch of the thread. Likewise, the total volume Tᵥ can be determined by the major diameter of the screw and the length of the shank. The volume of bone V_{b} to be occupied by the bone-receiving area can then be determined by subtracting the shank volume from the total volume Tᵥ. Based on these calculations, FIG. 5 illustrates a comparison of the shank volume, e.g., the amount of bone to be displaced, by four different screws, each having the same total volume Tᵥ. The dimensions used to calculate the total volume Tᵥ, the shank volume, and the volume of bone V_{b} for the different screws are set forth in Table 1 below.

TABLE 1

| | TAPER | | MAJOR | | |
|---|---|---|---|---|---|
| BONE SCREW | ANGLE | MINOR DIAMETER | DIAMETER | LENGTH | PITCH |
| Conventional | 0° | 4.5 mm | 7 mm | 31.5 mm | 3 mm |
| Full Tapered | 5.0° | 4.5 mm to 1.75 mm | 7 mm | 31.5 mm | 6.35 mm |
| Half Tapered | 11.6° | 4.5 mm to 1.75 mm | 7 mm | 31.5 mm | 6.35 mm |
| Quarter Tapered | 17.1° | 4.5 mm to 1.75 mm | 7 mm | 31.5 mm | 6.35 mm |

As shown in Table 1, each of these bone screws has a major diameter of about 7 mm and a length of about 31.5 mm, which results in a total volume Tᵥ of about 1212 mm³. As shown in FIG. 5, the conventional screw has a shank volume of 650 mm³, and thus will displace about 53.6% of the total volume Tᵥ. Since the shank volume is 650 mm³, the estimated volume of bone V_{b} occupied by the bone-receiving area will be 562 mm³ (1212 mm³-650 mm³), which is about 46.4% of the total volume Tᵥ. The bone screws of the present invention, on the other hand, will only displace 36.6% (444 mm³) of bone with a full taper, 33.2% (403 mm³) with a half taper, and 32.4% (393 mm³) with a quarter taper. As a result, the bone-receiving area 38 of the bone screws of the present invention will occupy 63.4% (768 mm³) of bone with a full taper, 66.8% (809 mm³) with a half taper, and 67.6% (819 mm³) with a quarter taper. Accordingly, the bone screws of the present invention displace a relatively small volume of bone, and occupy a relatively large volume of bone, thus causing less damage to the bone, while increasing the pull-out strength and the flexibility of the bone screw, and reducing the insertion torque.

In use, the bone screw 10, 100, 200, 300 is driven into bone, such as cortical or cancellous bone, using a driver tool that mates with the hexagonal socket 124, 224, 324 in the head of the screw. As the screw 10, 100, 200, 300 is inserted into the bone, the threads 16a, 16b, 116, 216, 316 will cut through the bone in a helical pattern such that the bone-receiving area 38, 138, 238, 338 between the threads 16a, 16b, 116, 216, 316 will be filled with bone. This will prevent the screw 10, 100, 200, 300 from being pulled out of the bone, and will reduce the amount of damage to the bone surrounding the screw 10, 100, 200, 300, as less bone needs to be displaced to implant the screw 10, 100, 200, 300. The relatively small minor diameter d₁, x₁, y₁, z₁ also provides sufficiently flexible to the screw to allow for load sharing across flanks of the threads 16a, 16b, 116, 216, 316, and to prevent the screw head 12, 112, 212, 312 from breaking off during insertion.

In the event that the bone screw must be removed, a revision screw can be provided to replace the removed bone screw. Typically, bone screws have threads that extend in a particular direction. As a result, when the screw is implanted, the screw will carve out an area of bone that corresponds to the direction of the threads. When the screw is removed, it can be difficult to insert another screw at the same location, as a certain amount of bone has already been removed and thus there is less bone available to engage with the new screw. Conventional methods require the use of a bone screw having a major diameter that is greater than the major diameter of the original, now removed screw. The screw of the present invention, however, can also be formed as a revision screw having threads extending in a reverse direction, thus allowing the screw to be implanted in a reverse direction. As a result, insertion of the revision screw will engage bone that was not carved out by the original screw, since the relatively small minor diameter of the screws according to the present invention remove less bone during implantation.

The bone screw according to the present invention can be made from any biocompatible material, including biocompatible metals and polymers. It is also contemplated that the bone screw can equally comprise bioabsorbable and/or biodegradable materials. Suitable materials include, but are not limited to, all surgically appropriate metals including titanium, titanium alloy, chrome alloys and stainless steel, and non-resorbable non-metallic materials such as carbon fiber materials, resins, plastics and ceramics. Exemplary materials include, but are not limited to, PEAK, PEEK, PEK, PEKK and PEKEKK materials net or reinforced with, for example, carbon fibers or glass fibers. A person skilled in the art will appreciate that any number of a wide variety of materials possessing the mechanical properties suitable for attachment with bone can be used.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A bone screw, comprising:
a head;
a shank having a major diameter, a minor diameter, and a length extending between a proximal end and a distal end; and
first and second helical threads having a root and a crest, the threads extending around the length of the shank and defining a thread thickness extending between proximal and distal facing flanks, the thread thickness of each thread adjacent the root of the threads being equal to or greater than the minor diameter of the shank along at least a portion of the length of the shank.

2. The bone screw of claim 1, wherein the thread thickness adjacent the root of the threads is equal to or greater than a minor diameter of the shank at a distal portion of the shank.

3. The bone screw of claim 1, wherein the minor diameter of the shank remains substantially constant along the length of the shank.

4. The bone screw of claim 1, wherein at least a portion of the minor diameter of the shank decreases in a proximal-to-distal direction to form a tapered portion.

5. The bone screw of claim 1, wherein the crest of the threads has a width extending between the proximal and distal facing flanks that remains substantially constant.

6. The bone screw of claim 5, wherein the crest of the threads is substantially flat.

7. The bone screw of claim 5, wherein the width of the crest is about 0.2 mm.

8. The bone screw of claim 1, wherein the threads have a pitch of about 6 mm.

9. The bone screw of claim 1, wherein the proximal and distal flanks of the threads converge toward one another at an angle from the root to the crest of the threads.

10. The bone screw of claim 9, wherein the proximal and distal flanks converge toward one at substantially the same angle.

11. The bone screw of claim 1, wherein the thread thickness at the root of the threads is substantially constant along the length of the shank.

12. The bone screw of claim 11, wherein the proximal and distal facing flanks converge toward one another at an outer-most crest of each thread to form a beveled edge.

13. The bone screw of claim 1, wherein the threads define a bone-receiving area between adjacent flanks that is adapted to seat a volume of bone when the shank is disposed within bone, the volume of bone being at least about 50% of a total volume defined by the major diameter and the length of the shank.

14. The bone screw of claim 13, wherein the bone-receiving area has a volume that is at least about 60% of the total volume.

15. The bone screw of claim 1, wherein the major diameter is substantially constant along a substantial portion of the length of the shank.

16. The bone screw of claim 15, further including a pointed apex formed at the distal end of the shank.

17. The bone screw of claim 16, wherein the pointed apex is formed by the threads.

18. A bone screw, comprising:
a head having a driver-receiving element formed thereon;
a shank formed from first and second helical threads offset approximately 180° from one another and extending distally from the head around a core defining a minor diameter, the shank having a size effective to displace a volume of bone, when the shank is disposed within bone, that is less than about 50% of a total volume defined by a major diameter and a length of the shank; and
a pointed tip formed at a distal end of the shank.

19. The bone screw of claim 18, wherein the minor diameter remains substantially constant.

20. The bone screw of claim 18, wherein at least a portion of the minor diameter decreases in a proximal-to-distal direction to form a tapered portion.

21. The bone screw of claim 18, wherein the threads define a thread thickness that is substantially constant along a length of the shank at a root of the threads.

22. The bone screw of claim 18, wherein the major diameter remains substantially constant along a substantial length of the shank.

23. The bone screw of claim 18, wherein the threads each include proximal and distal facing flanks having a first portion adjacent a root of the threads that is substantially parallel to each other, and a second portion at a crest of the threads that converges toward each other to form a beveled edge.

24. The bone screw of claim 18, wherein the threads each include proximal and distal facing flanks that converge toward one another at an angle from a root to a crest.

25. The bone screw of claim 24, wherein the proximal and distal flanks converge toward one another at substantially the same angle.

26. The bone screw of claim 18, wherein the threads include a flat crest defining a width that remains substantially constant along a length of the shank.

27. The bone screw of claim 18, wherein the screw has right-handed threads.

28. The bone screw of claim 18, wherein the screw has left-handed threads and is a revision screw.
